(19) United States
(12) Patent Application Publication
(10) Pub. No.: US 2020/0138616 A1
(43) Pub. Date: May 7, 2020

(54) DEVICE FOR USE AS A THERAPEUTIC AID FOR THE THERAPEUTIC TREATMENT OF A SYMPTOM OF PARKINSON'S DISEASE

(71) Applicant: REHA-TEAM PERICK GMBH, Steinfurt (DE)

(72) Inventor: Jürgen PERICK, Steinfurt (DE)

(73) Assignee: REHA-TEAM PERICK GMBH, Steinfurt (DE)

(21) Appl. No.: 16/619,811

(22) PCT Filed: Jun. 6, 2018
(86) PCT No.: PCT/DE2018/100539

§ 371 Date: Dec. 5, 2019

(30) Foreign Application Priority Data
Jun. 8, 2017 (DE) 20 2017 002 987.1
Aug. 21, 2017 (DE) 10 2017 119 103.8

(51) Int. Cl. A61F 5/02; A61H 1/00

(52) U.S. Cl. CPC A61F 5/026; A61H 2201/1652; A61H 2205/084; A61H 1/008

(57) ABSTRACT

The invention refers to a device (1) for use as a therapeutic aid for the therapeutic treatment of a symptom of Parkinson's and its use as device for therapeutic treatment.

The invention provides for the device (1) to have at least two pressure transducers (2) that can be arranged and set up to deliver pressure impulses to contact points on the left and right pectoral muscles of a patient.

The invention refers to a device for use as a therapeutic aid for the therapeutic treatment of a symptom of Parkinson's disease and its application.

### STATE OF THE ART

One of the main symptoms of Parkinson's disease is rigidity or muscle stiffness, which manifests itself, among other ways, in the form of a stooped posture, also known as camptocormia.

Swallowing disorders and thus fundamental food and fluid intake problems, as well as breathing problems, are further side effects of camptocormia.

On the one hand, camptocormia is treated indirectly through pharmaceutical treatment of the underlying disease, and on the other hand, directly by means of the botulinum toxin, physical therapy, occupational therapy, acupuncture or massage. DBS, deep brain stimulation, is also a known treatment.

As a whole, treatment of the camptocormia symptom of Parkinson's disease is considered difficult.

### DISCLOSURE OF THE INVENTION

The purpose of the present invention is the disclosure of a new therapeutic aid for the therapeutic treatment of a symptom of Parkinson's disease, specifically for camptocormia.

According to the invention, we refer to a device for use as a therapeutic aid for the therapeutic treatment of a symptom of Parkinson's which has at least two pressure transducers, which can be arranged to deliver pressure impulses to contact points on a patient's left and right pectoral muscles and can be set up to deliver pressure impulses, i.e. through which pressure impulses can be delivered to contact points on the left and right pectoral muscles.

The advantage of such a device is that such a delivery of pressure impulses to certain pectoral muscle regions, specified here as contact points, can be activated at any time, whereby through the repeated effect of the pressure impulses on the pectoral muscle contact points, the forward-bending muscles are able to relax in many cases.

An advantage of such a device is the possibility, for example, of supplementary, comparatively gentle support of camptocormia treatment with an aid having few, if any, material side effects, especially compared to the use of pharmaceutical active ingredients and relatively less need of medical staff.

The repeated stimulation of contact points on the pectoral muscles, specifically the repeated exertion of pressure impulses on certain regions of the pectoral muscles, especially by pressure transducers in the form of harness straps, has shown the surprising effect of counteracting camptocormia.

Advantageous in such a device are the means to position the pressure transducers on contact points in the upper pectoral muscle area, as the contact points can be on different places in the individual patients.

In addition, the patient can wear both the device and the positioning aids on his body. With this design, the patient is able to wear the device for a longer period of time, regardless of a stationary or ambulatory treatment, over longer effective periods.

In this case, results from an initial internal study performed on a small group of about 25 patients showed surprisingly positive results. Thus, it could be determined that symptoms were relieved in 60-70% of subjects, in many cases reflected in noticeably better straight posture. Also, improved breathing owing to rib cage expansion and better swallowing of food and fluids could be observed. The positive results achieved in the small, internal group of subjects led to the planning of clinical studies.

The positioning aids can be designed, for example, in the form of a special garment or as a harness, or the pressure transducers can be designed in the form of harness straps, for example. Such a design allows the patient not only to be mobile, but also to be discrete, since the device can also be worn under the clothes if it is to be used accordingly.

The pressure transducers are advantageously designed in form of harness straps. A further advantage of the straps is their curved shape. In this case, the straps can be designed in the form of shoulder straps to enclose the shoulder joint.

A curved shape very much increases the likelihood that the contact points responsible for muscle relaxation will be covered. With straight straps lacking a curved shape, this can only be accomplished with great difficulty or not at all reliably. The curved shape of the straps makes them also easy to be guided around a shoulder joint while offering good support. Here, the straps can be separated or designed in such a way that a loop formed with the straps to enclose the shoulder joint can be lengthened or expanded to a size of at least 100 cm.

Such a design offers the advantage that patients generally suffering from muscle stiffness can slip the strap over the shoulder without great difficulty. It is advantageous to lengthen the loop to a size of 100 cm to 120 cm, so that the loop can also be guided around an almost fully extended arm. To reduce the loop width, the device advantageously has locking mechanisms to lock the tightened straps. Another advantage is that the straps can have a grasping end, which can be easily grasped if the loop is in a widened state. Since the pinch grip is often difficult for Parkinson's patients, an easily-grasped grasping end at the end of a strap is at least approx. 10 cm long. So the loop is not accidentally opened when being widened, the device can be fitted with a safety device through which the provision of a grasping end can be protected.

The protection can be fitted in the form of a slip guard, especially in the form of a rivet, clamp or button, which would prevent the end of the strap from slipping through a buckle when placed on a strap, for example. Alternatively, the safety protection can also be fitted as a loop, handle or snap hook.

If the straps guided around the shoulder joints have a curved shape and an approximate width of 5-8 cm, then they can more than likely cover the contact points that will reach the muscles to relax them. To reach the contact points with more certainty, the device can advantageously be fitted with aids to precisely adjust the distance of the straps, for example a cross belt, through which the distance of the straps can be adjusted on the chest. To achieve this, the cross belt can be connected to the straps. In an advantageous design, the height of the cross belt connections are adjustable with the straps.

Conveniently, the device is designed in such a way that the patient hardly notices it when used on the shoulders and back or, ideally, potentially not at all after an adaptation phase. To achieve this, the straps can be fitted advantageously in such a way they sit flat on the shoulder and back area. In an advantageous design of the invention, the straps have an approximate width of 5-8 cm in the shoulder and back area.

In the pectoral muscle area as well as in the shoulder and back area, the contact surfaces of the device can have similar, equal or even different widths.

To ensure the effectiveness of the device, it is necessary that the straps adjusted on the contact points of the pectoral muscles do not slip. This can be accomplished, at least partially, by fitting the straps flat against the skin in the back area. Advantageously, the straps in the back area have a certain stiffness, chosen in such a way that they hardly hinder, or do not hinder at all, movements around the body's longitudinal axis, especially turns, and around the body's transverse axis, especially flexions, and noticeably hinder any compression along the body's transverse axis.

For this purpose, the straps in the back area can be fitted flat, flat and one-dimensionally connected to one another, and/or with several layers. In a special embodiment, the straps can have, for example, an insert that interconnects the straps and stiffens the distance and/or angle between the straps.

The insert's advantageous quality, owing to the choice of shape and/or material, also allows for deformation in the stiffening directions with moderate effort, which is somewhat noticeable when wearing it. Possible embodiments of the insert can be, for example, a stiff textile fabric or a thin plastic insert. As an alternative to an insert, the strap material itself can also have these properties.

A backside pocket arranged on the back side contributes to the device's convenient design. Thereby, the rear position of the backside pocket is to be understood as a localization, which means that the backside pocket is arranged on the patient's back when he carries the device along. The arrangement is advantageous because owing to the positioning and/or shaping of the backside pocket, the development of contact points or pressure points on the patient's back is prevented or minimized.

Such a design not only makes it more comfortable to wear, but also prevents the influence of additional stimuli on the patient which deflect the stimuli coming from the pressure transducers and thereby lessen and/or jeopardize therapeutic success.

Advantageously, the backside pocket is connected to the straps for centralized positioning on the back. For instance, it can be sewn together with them to accomplish this.

Another advantage is that the length of the straps are adjustable in such a way that the backside pocket can be positioned on a patient centrally below the shoulder blades. To accomplish this, the straps can be joined in the shape of a V, a U or an arch and therefore specify the vertical localization on the back. For this, the straps conveniently have an approximate width of 5-8 cm in this area to support lying flat on the body, thereby lessening the danger of twisting or slipping.

A further advantage of the invention's design is the straps' V-shape arrangement and 45 to 55 degree opening angle, particularly 50 degrees. For average proportioned patients, this angle allows the straps to be well positioned on the shoulder, regardless of their size, so the contact points on the pectoral muscles can be reached and also allows for the desired positioning of a backside pocket below the shoulder blades.

In the area above the shoulder, the straps are advantageously bent slightly inward towards the sternum. The straps have an advantageous mean curvature radius R1 of 40-45 cm, especially of 43 cm, in the shoulder area.

In the area above the breast, the straps have a beneficial slight outward bend covering the pectoral muscle, partially enclosing the shoulder joint. The straps have an advantageous mean curvature radius R2 of 25-30 cm, especially of 28 cm, in the pectoral muscle area.

The straps are advantageously curved and S-shaped. According to the preceding designs, the straps have advantageously both an inward and outward curvature. From the rear, a view of an open harness stretched flat shows the course of the left strap from the back to the chest creating an “S”-shape and the course of the right strap from the chest to the back an “S.”

A further advantage is that the length of the strap section running along the back is adjustable. This can be necessary to ensure the right positioning of a curved portion of the strap on the pectoral muscles even in very small or very portly patients. Alternately, the device can be connected with different-sized straps. To meet the different size requirements of patients, it may also be necessary to select radius R1 from a 20-35 cm range, and radius R2 from a 35-50 cm range.

Another benefit includes the flat and/or pillow-shaped design of the backside pocket, whereby it does not exceed a height of 3 cm, to the patient's benefit. In an advantageous design, the backside pocket is less than 2 cm high. Such a design allows the device to be worn relatively comfortably and inconspicuously, with the backside pocket under the clothes.

The backside pocket is designed to receive a flat weight, which can be firmly connected to the backside pocket, such as by sewing it in.

Advantageously, however, the backside pocket is alternately configured to accommodate between one to three flat weights of 0.25 kg to 0.75 kg, 0.5 kg to 1.5 kg, or 0.5 kg to 2 kg as needed.

The exerted force of the flat weights is suitable for exercising traction on the straps of the device which conduct the weight in the form of pressure on the patient's body. In doing so, the patient's inertia-based movements lead to pressure fluctuations which manifest themselves in form of pressure impulses when walking, for example. Thus, in this advantageous design, the pressure transducers of the device can be driven by the patient's movement. Alternatively or additionally, the device can have an electric pressure transducer. Moreover, the device can have an energy source shaped like a flat lead battery, for example, which can be fitted alternatively or in addition to a weight in the backside pocket.

Alternatively, the backside pocket being filled with one two or three weights allows the individual patient to adjust the intensity of the impulses, whereby the fitting of the weighs from 0.25 kg to 0.75 kg, 0.5 kg to 1.5 kg, and 0.5 kg to 2 kg, together with the various combination options, covers a useful weight range. This comparatively narrow weight range is generally sufficient for the effectiveness of the device, as the straightened posture the device works towards is not caused by the mechanical leverage of the weights pulling backward, but by the pressure impulses delivered to the pectoral muscles. In an advantageous design, the weights are fitted in a set of 0.5 kg, 1 kg, and 1 kg. Another benefit is that they have the exact dimensions of the backside pocket and rounded corners and are sealed in foil. The precise adjustment to the dimensions of the backside pocket prevents the weights from moving in the backside pocket and therefore prevents the accidental generation of interference pulses.

Also advantageously, the backside pocket of the device can have a roughly rectangular base surface, which can conveniently be 15-19 cm wide×17-22 cm long, whereby the longitudinal side is oriented parallel to the patient's spine when in use. Advantageously, the external dimensions of the backside pocket are 17×19.5 cm. A backside pocket with such dimensions offers the advantage of being small enough to be used in different-sized patients yet large enough to receive flat weights with a sufficiently large mass.

As an alternative to a rectangular, flat-fit backside pocket, it can also be designed to adapt to the body's anatomy and have, for example, a curvature for that purpose. Furthermore, it can have a recess or groove for the spine and/or basic shapes other than a rectangle.

In an advantageous design of the device, the backside pocket has a first viscoelastic foam material on the side facing the patient's back. It can be advantageously dimensioned and arranged in such a way that the weights can be fixed in place by the foam material in a partially filled backside pocket. In this case, the viscoelastic foam material is so advantageously dimensioned and arranged that in a relaxed state it largely fills the interior of the backside pocket.

In another advantageous design of the device, the backside pocket has a second viscoelastic foam material arranged in such a way that, in order to prevent contact points or pressure points, a clamping force exerted by the backside pocket weights on the patient's back is flatly distributed, also with a view of suppressing the accidental generation of interference pulses.

The device is designed with the advantage that the first and second viscoelastic foam material are fitted as one single piece.

Another advantage is that the backside pocket can be closed. Such a closure prevents the weights from slipping out of the backside pocket, especially when bending over. Here, the closure can advantageously be a Velcro fastener. With regard to the beneficial design of the Velcro fastener, it is arranged on the interior of the backside pocket in such a way that the lateral edge of the Velcro fastener lies directly on a weight that can be held in the pocket.

Furthermore, the device can advantageously have a mechanical impulse converter suitable for converting a flat pressure impulse emitted by a pressure transducer to one or several selective pressure impulses. In this way, the pressure impulses can be strengthened or the weights reduced. Here, a pressure impulse that activates only selectively is understood to be one acting on a surface of a few square millimeters to several square centimeters. Furthermore, the impulse converter can be fitted in the form of one or several knobs that can be arranged below a pressure transducer.

Moreover, the invention provides for the use of a device with two pressure transducers for delivering pressure impulses to a patient's left and right pectoral muscles as a therapeutic device, especially for a patient who suffers from Parkinson's disease, whereby—as is evident from preceding designs—the effect of the device is rooted in the device itself.

Furthermore, the invention provides for the use of a device as a therapeutic aid for the therapeutic treatment of a symptom of Parkinson's that has at least two pressure transducers, which can be arranged on a patient's left and right pectoral muscles to deliver pressure impulses to contact points and that can be set up to deliver pressure impulses.

With an advantageous embodiment, a device for use as a therapeutic aid for the therapeutic treatment of a symptom of Parkinson's is a therapeutic device for treating camptocormia, shaped like a camptocormia backpack, for example.

With an advantageous embodiment, the invention refers to a camptocormia backpack with curved shoulder harnesses for the therapeutic support of Parkinson's therapy, whereby the shoulder harnesses—also referred to as straps—have a curved shape, adjustable length, and the camptocormia backpack has a flat backside pocket to receive flat weights and a transverse belt on the chest for precisely adjusting the straps, and whereby for the purpose of achieving a therapeutic effect, the camptocormia backpack is put on as follows:
The flat backside pocket is centrally positioned on a patient's back above the strap adjustment and without touching the shoulder blades, and on the side of the chest, the distance of the curved straps is adjusted with the transverse belt,
whereby the curved straps cover the contact points on the pectoral muscles
and pressure impulses are delivered to the contact points as a result of the patient's movement.

In other embodiments, the camptocormia backpack can have more design features than the ones described above for use as a therapeutic aid for the therapeutic treatment of a symptom of Parkinson's.

### ILLUSTRATIONS

The invention will be described in more detail below by means of preferable embodiments, making reference to the attached schematic drawings.

They show

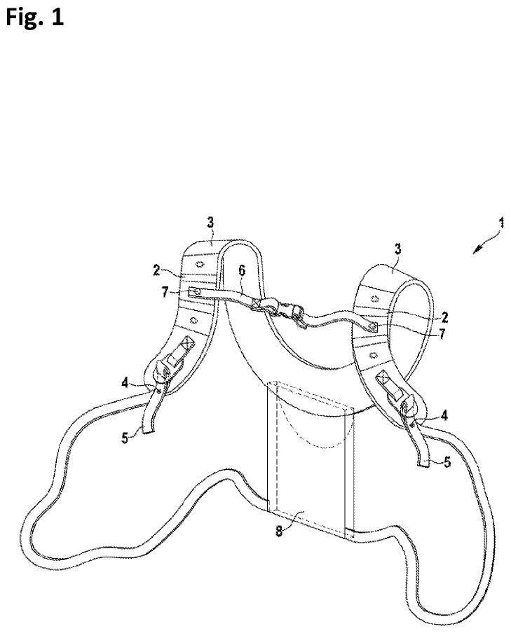

FIG. 1 A sketch of a camptocormia backpack as the preferable design of a device for use as a therapeutic aid for the therapeutic treatment of a symptom of Parkinson's;

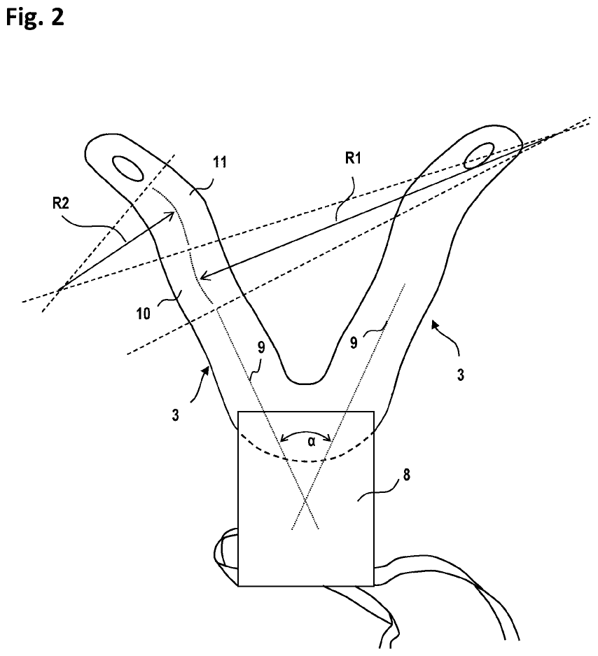

FIG. 2 A sketch of a camptocormia backpack with extended curved-shaped straps.

FIG. 1 shows a sketch of a camptocormia backpack 1 in perspective.

A camptocormia backpack 1 as the preferable design of a device 1 for use as a therapeutic aid for the therapeutic treatment of a symptom of Parkinson's is evident in FIG. 1, whereby the device 1 has two pressure transducers 2, which can be arranged and set up to deliver pressure impulses to contact points on a patient's left and right pectoral muscles.

Here, the pressure transducers 2 are fitted in the form of straps 3 of a harness. To adjust the distance of the straps 3, the harness has aids 6, whereby the aids 6 are height-adjustable for adjusting the spacing of the straps 3 with the help of aids 7. The straps 3 have a curved shape and are fitted in the form of shoulder straps to enclose the shoulder joint.

A loop created with the straps 3 to envelop the shoulder joint is expandable to a length of at least 100 cm. The straps 3 have a grasping end 5, easily grasped when the loop has been widened, i.e. when it is about 10 cm long. The camptocormia backpack 1 has a safety device 4, through which the provision of the grasping end 5 can be protected.

Also evident in FIG. 1, the camptocormia backpack 1 has a flat backside pocket 8 situated on the backpack less than 3 cm high. The backside pocket 8 is shaped in a way to prevent the development of individual contact points on the patient's back, especially on the shoulder blades. The length of the straps 3 can be adjusted in such a way that the backside pocket 8 is centrally positioned below the patient's shoulder blades.

The fitted backside pocket 8 has a roughly rectangular base surface that is 17 cm wide and 19.5 cm long, allowing it to alternately accommodate one to three 0.5 kg, 1 kg, and 1 kg weights as needed.

On the side facing the patient's back, the backside pocket 8 has a viscoelastic foam material (not shown). The viscoelastic foam material is dimensioned and arranged on the side facing the patient's back in such a way that the weights are fixed in place by the foam material in a partially filled backside pocket.

If there are no weights in the backside pocket 8, the viscoelastic foam material in a relaxed state largely fills the interior of the backside pocket 8, i.e. almost completely. Furthermore, the foam material is arranged in such a way that, to prevent contact points, a clamping force exerted by the weights of the backside pocket 8 is flatly distributed.

FIG. 2 shows a sketch of a camptocormia backpack with extended straps, whereby the straps have a curved shape.

The straps 3 are arranged in a V-shape towards one another and have an opening angle α between 45 degrees and 55 degrees, specifically of 50 degrees. Here, a fictitious intersection of the straps' center lines 9 lies roughly centrally in the backside pocket 8. In an area above the shoulder 10, the straps 3 are advantageously curved slightly inwards in the direction of the sternum. The median curvature radius R1 of the straps 3 in this area 10 is between 40 cm and 45 cm, specifically 43 cm. In an area above the chest 11, the straps 3 are curved slightly outwards, partially enclose the shoulder joint and cover the pectoral muscle. In the area 11, the straps 3 have a median curvature radius R2 between 25 cm and 30 cm, specifically of 28 cm. Furthermore, as is evident from FIG. 2, the straps are S-shaped and curved. In the image depicting an open harness stretched flat, from the rear perspective, the course of the left strap 3 creates an “S”-shape from the back to the chest, i.e. in FIG. 2 from bottom to top, and the course of the right strap 3 from the chest to the back, i.e. in FIG. 2 from top to bottom, likewise creates an “S”-shape.

1. Device (1) for use as a therapeutic aid for the therapeutic treatment of a symptom of Parkinson's, characterized in that the device (1) has at least two pressure transducers (2), which can be arranged and set up to deliver pressure impulses to contact points on a patient's left and right pectoral muscles.

2. Device (1) according to claim 1, characterized in that the pressure transducers (2) are fitted in the form of harness straps (3), that the harness has aids (6) to adjust the spacing of the straps (3) and the straps (3) have a curved shape and are fitted in the form of shoulder harnesses to enclose the shoulder joint.

3. Device (1) according to claim 2, characterized in that the straps (3) can be separated or a loop created with the straps (3) to enclose the shoulder joint and widened to a length of at least 100 cm.

4. Device (1) according to claim 2, characterized in that the straps (3) have one grasping end (5), which in the widened state creates an easily grasped loop out of the straps (3) to enclose the shoulder joint and the device has a safety device (4) through which the provision of a grasping end (5) can be protected.

5. Device (1) according to claim 2, characterized in that the device (1) has a flat backside pocket (8) situated on the back side with a height of less than 3 cm.

6. Device (1) according to claim 5, characterized in that the backside pocket (8) has been designed in a way to prevent the formation of individual contact points on the patient's back.

7. Device (1) according to claim 5, characterized in that the length of the straps (3) can be adjusted in such a way that the backside pocket (8) can be positioned centrally below the patient's shoulder blades.

8. Device (1) according to claim 5, characterized in that the backside pocket (8) is designed in the form of a pillow.

9. Device (1) according to claim 5, characterized in that the backside pocket (8) is fitted for alternately accommodating one to three flat weights of 0.25 kg to 0.75 kg, 0.5 kg to 1.5 kg, and 0.5 kg to 2 kg as needed.

10. Device (1) according to claim 5, characterized in that the backside pocket (8) has a rough rectangular base surface of 15 cm, a width of 19 cm and a length of 17-22 cm.

11. Device (1) according to claim 5, characterized in that the backside pocket (8) has a first viscoelastic foam material on the side facing the patient's back.

12. Device (1) according to claim 11, characterized in that the first viscoelastic foam material is dimensioned and arranged in such a way on the side facing the patient's back that the weights can be fixed in place in a partially filled backside pocket by the foam material.

13. Device (1) according to claim 11, characterized in that the first viscoelastic foam material largely fills the interior of the backside pocket (8) in a relaxed state.

14. Device (1) according to claim 11, characterized in that the device (1) has a second viscoelastic foam material arranged in such a way that a clamping force exerted on the back of the patient by the backside pocket (8) is flatly distributed to prevent contact points.

15. Device (1) according to claim 11, characterized in that the first and second foam material are fitted as one single piece.

16. Device (1) according to claim 1, characterized in that the device (1) has an impulse converter suitable for converting a flat pressure impulse delivered by a pressure transducer (2) to one or several selective pressure impulses.

17. Device (1) according to claim 16, characterized in that the impulse converter is fitted in the form of one or several knobs that can be arranged under a pressure transducer (2).

18. Set of three flat weights for combination with a device according to claim 5, whereby the flat weights are fitted in the form of rectangular and foil-sealed weight plates with rounded corners weighing 0.25 kg to 0.75 kg, 0.5 kg to 1.5 kg and 0.5 kg to 2 kg, adjusted for combination and retractable in the backside pocket (8) of the device (1) to generate pressure impulses on contact points on the left and right pectoral muscles of a patient.

19. Use of a device (1) with two pressure transducers (2) to deliver pressure impulses to contact points on the left and right pectoral muscles of a patient as a therapeutic device for Parkinson's patients.

20. Use of a device (1) according to claim 1 as a therapeutic aid for Parkinson's patients.
